(19) United States
(12) Patent Application Publication
(10) Pub. No.: US 2016/0361290 A1
(43) Pub. Date: Dec. 15, 2016

(54) PHARMACEUTICAL COMPOSITIONS FOR THE TREATMENT OF DISEASE AND/OR SYMPTOMS IN ARTHRITIS

(71) Applicant: GW Pharma Limited, Salisbury (GB)

(72) Inventors: Philip Robson, Salisbury (GB); Geoffrey Guy, Salisbury (GB)

(73) Assignee: GW Pharma Limited, Salisbury (GB)

(21) Appl. No.: 15/068,707

(22) Filed: Mar. 14, 2016

Related U.S. Application Data
(63) Continuation of application No. 11/628,814, filed on Aug. 16, 2007, now abandoned.

(30) Foreign Application Priority Data
Jun. 8, 2004 (GB) 0412753.6

(51) Int. Cl. A61K 31/352; A61K 36/185; A61K 9/00; A61K 31/05; A61K 47/10

(52) U.S. Cl. CPC A61K 31/352; A61K 31/05; A61K 47/10; A61K 9/0073; A61K 36/185; A61K 2236/15; A61K 2236/39; A61K 2236/51; A61K 2236/55

(57) ABSTRACT

The invention relates to the use of a combination of cannabinoids for the treatment of pain, inflammation and/or disease modification in arthritis. Preferably the cannabinoids are selected from cannabidiol (CBD) or cannabidivarin (CBDV) and delta-9-tetrahydrocannabinol (THC) or tetrahydrocannabinovarin (THCV). More preferably the cannabinoids are in a predefined ratio by weight of less than or equal to 19:1 of CBD or CBDV to THC or THCV.

The present invention relates to the use of a combination of cannabinoids for the treatment of pain, inflammation and/or disease modification in arthritis. Preferably the cannabinoids are selected from cannabidiol (CBD) or cannabidivarin (CBDV) and delta-9-tetrahydrocannabinol (THC) or tetrahydrocannabinovarin (THCV). More preferably the cannabinoids are in a predefined ratio by weight of less than or equal to 19:1 of CBD or CBDV to THC or THCV.

### BACKGROUND TO THE INVENTION

Arthritis is a painful condition of the joints. There are different types of the disease yet all cause pain and inflammation of the joints and are often degenerative in nature. Some of the most common types of arthritis are osteoarthritis and rheumatoid arthritis.

Osteoarthritis is a disease that affects the joints of around 8 in 10 people over the age of 50. Osteoarthritis is caused by the joint cartilage becoming thin and uneven over time and can in some cases wear out completely. In addition to the wearing out of the joints, the joint capsule can become thicker and in consequence there is an increase in the amount of synovial fluid that is generated. This in turn causes the joint to swell. Bony spurs may also grow in the affected area causing inflammation in the affected tissues. Osteoarthritis can involve all joints in the body, but is most commonly found in the fingers, knees, hips and spine.

Rheumatoid arthritis is a systemic disease, which can affect the entire body and is one of the most common forms of arthritis. It is characterised by inflammation of the membranes that line a joint, which in turn causes pain, stiffness, warmth, redness and swelling to the area. The small joints of the fingers and hands are most seriously affected but the condition can spread to involve the wrists, elbows, shoulders and other joints. The inflamed joint lining can also invade and damage bone and cartilage when inflammatory cells release enzymes that are able to digest bone and cartilage. The inflamed joint can lose its shape and alignment, resulting in pain and loss of movement. It is typically chronic and can flare-up at intervals.

In addition to the pain and inflammation experienced in the affected joints, rheumatoid arthritis can cause loss of appetite and weight, lethargy, muscle and tendon pain, fever, lumps under the skin (rheumatoid nodules) and severe eye inflammation. There are many complications including anaemia, pericarditis, vasculitis and Raynaud's phenomenon.

The cause of rheumatoid arthritis is not yet known. However, it is known that rheumatoid arthritis is an autoimmune disease. The body's natural immune system does not operate as it should, resulting in the immune system attacking healthy joint tissue and causing inflammation and subsequent joint damage. The disease could be triggered by an infection in some people who have an inherited tendency for the disease that prompts the immune system to form damaging aggregates of antigen and antibody immune complexes.

Early in the disease, people may notice general fatigue, soreness, stiffness and aching. Pain and swelling may occur in the same joints on both sides of the body and will usually start in the hands or feet. Rheumatoid arthritis affects the wrist and many of the hand joints, but usually not the joints that are closest to the fingernails (except the thumb). Rheumatoid arthritis can also affect elbows, shoulders, neck, knees, hips and ankles. It tends to persist over prolonged periods of time, and over time, the inflamed joints may become damaged.

Treatment of rheumatoid arthritis is limited to the control of inflammation and the relief of pain by means of rest, splinting of the inflamed joint, physiotherapy, and the use of anti-inflammatory and pain killing drugs. The treatment methods focus on relieving pain, reducing inflammation, stopping or slowing joint damage, and improving the patient's well being.

The current medications that are provided to patients with rheumatoid arthritis can be divided into two groups;
1. Symptomatic medications, such as non-steroidal anti-inflammatory drugs (NSAIDs) and aspirin, analgesics, and corticosteroids. These drugs help reduce joint pain, stiffness and swelling. Symptomatic medications may be used in combination with disease-modifying anti-rheumatic drugs.
2. Disease-modifying anti-rheumatic drugs (DMARDs), include low doses of methotrexate, leflunomide, D-Penicillamine, sulfasalazine, gold therapy, minocycline, azathioprine, hydroxychloroquine (and other antimalarials), cyclosporine and biologic agents.

In addition to drug therapy, treatment most often involves some combination of exercise, rest, joint protection, and physical and occupational therapy. Surgery can be an option for joints that are severely damaged and painful. A balance of rest and exercise can help conserve energy and maintain a range of motion and use of the joints.

The use of cannabis as a medicine has long been known and during the 19^{th} Century preparations of cannabis were recommended as a hypnotic sedative which were useful for the treatment of hysteria, delirium, epilepsy, nervous insomnia, migraine, pain and dysmenorrhoea.

Until recent times the administration of cannabis to a patient could only be achieved by preparation of cannabis by decoction in ethanol, which could then be swallowed or by the patient inhaling the vapours of cannabis by smoking the dried plant material. Recent methods have sought to find new ways to deliver cannabinoids to a patient including those which bypass the stomach and the associated first pass effect of the liver which can remove up to 90% of the active ingested dose and avoid the patient having to inhale unhealthy tars and associated carcinogens into their lungs.

Such dosage forms include administering the cannabinoids to the sublingual or buccal mucosae, inhalation of a cannabinoid vapour by vaporisation or nebulisation, enemas or solid dosage forms such as gels, capsules, tablets, pastilles and lozenges.

In 1988 a study was undertaken in order to determine the analgesic and anti-inflammatory activity of various cannabinoids and cannabinoid pre-cursors. Oral administration of CBD was found to be the most effective at inhibition of PBQ-induced writhing in mice. THC and CBN were found to be least effective at reducing analgesia and inflammation (Formukong et al., 1988).

Holdcroft et al. have shown that cannabinoids can have analgesic and possible anti-inflammatory properties. Administration of 50 mg of THC to a patient with Mediterranean fever resulted in a highly significant reduction in the amount of analgesia that the patient required (Holdcroft et al., 1997a).

A follow-on publication by the same authors examined the oral administration of oil of cannabis. The capsules containing 5.75% THC, 4.73% CBD and 2.42% CBN were administered to a patient with familial Mediterranean fever. During the 3 weeks of active treatment there was a decrease in the amount of escape medication (morphine) required by the patient (Holdcroft et al., 1997b). There were no changes in the measured inflammatory markers.

It has previously been shown by Feldmann et al. (International patent application WO 99/52524) that pure CBD can be used to treat inflammatory diseases such as rheumatoid arthritis or Crohn's disease. Inflammatory diseases involve a complex interaction between several components such as Interleukins, TNF-α and nitric oxide. The data presented by Feldmann et al. describes the inhibition of TNF-α and nitric oxide production by CBD. This cannabinoid was also shown to suppress arthritis in a dose dependant manner in a collagen induced arthritis model in mice.

There is considerable literature concerning the immune modulating effects of constituents of cannabis a review of these was undertaken by Klein (Klein, 1998).

The use of different ratios of cannabinoids such as THC or CBD or their propyl variants, tetrahydrocannabinovarin (THCV) and cannabidivarin (CBDV), in the treatment of different diseases and conditions has previously been described by the applicant in their UK patent application GB2377633.

Specific ratios of THC and CBD or THCV and CBDV were reported to have been useful in the treatment or management of specific diseases or medical conditions. The following table details some of these areas.

| Product Group | | |
|---|---|---|
| Area | Ratio THC:CBD | Target Therapeutic |
| High THC | >95:5 | Cancer pain; |
| | | Migraine; |
| | | Appetite stimulation. |
| Even ratio | 50:50 | Multiple sclerosis; |
| | | Spinal cord injury; |
| | | Peripheral neuropathy; |
| | | Neurogenic pain. |
| Broad ratio CBD | <25:75 | Rheumatoid arthritis; |
| | | inflammatory bowel |
| | | disease. |
| High CBD | <5:95 | Psychotic disorders |
| | | (schizophrenia); |
| | | Epilepsy; |
| | | Movement disorders; |
| | | Stroke; |
| | | Head injury; |
| | | Disease modification |
| | | in rheumatoid |
| | | arthritis and other |
| | | inflammatory |
| | | conditions; |
| | | Appetite suppression. |

Formulations containing specific, defined ratios of cannabinoids may be formulated from pure, synthetic cannabinoids or from extracts derived from the cannabis plant in combination with pharmaceutical carriers and excipients.

A major disadvantage with the currently available drug therapies to treat arthritis is that the patient often has to take a combination of drugs in order to treat the symptoms of the disease such as the pain and associated inflammation, and at the same time the patient has to take a drug in order to modify the disease.

At present there are no known medications to treat the symptoms of pain and inflammation and at the same time act as disease modifying anti-rheumatic drugs.

Surprisingly it has been found that the use of a cannabis based medicine extract that contains approximately equal amounts of the cannabinoids delta-9-tetrahydrocannabinol (THC) and cannabidiol (CBD) can be used to both modify rheumatoid arthritis disease and treat the symptoms of pain and inflammation caused by the disease.

An important benefit of the use of the medication described in the present invention is that both the disease and the symptoms of the disease can be treated by the same medication. This in turn has numerous benefits which include a greater degree of flexibility for the patient as they will have to adhere to a less strict drug regime, the patient is also likely to experience less side effects as there will be less potentially harmful interaction between combined drug therapies.

### SUMMARY OF INVENTION

According to the first aspect of the present invention there is provided the use of a combination of cannabinoids x and y, where x is selected from the group consisting of cannabidiol (CBD) and cannabidivarin (CBDV) and where y is selected from the group consisting of delta-9-tetrahydrocannabinol (THC) and tetrahydrocannabinovarin (THCV), in the manufacture of a pharmaceutical formulation for use in the treatment of arthritis wherein the ratio of x:y by weight is less than or equal to 19:1.

Preferably the treatment of arthritis is the treatment of osteoarthritis or rheumatoid arthritis.

One embodiment of the invention provides a combination of cannabinoids for use in the treatment of one or more of the symptoms of pain, inflammation or lack of sleep in arthritis. Preferably there is provided a combination of cannabinoids for use in disease modification in arthritis. More preferably there is provided a combination of cannabinoids for use in the treatment of one or more of the symptoms and in disease modification of arthritis.

In one embodiment the ratio of cannabinoids x:y is less than or equal to 19:1, more preferably the ratio of x:y is less than or equal to 17:1 through to less than or equal to 3:1 in integers of 2. More preferably the ratio of x:y is less than or equal to 2.5:1 through to less than or equal to 1.25:1 in integers of 0.25. Most preferably the ratio of x:y is substantially 1:1, particularly 0.93:1.

Preferred combinations of cannabinoids include CBD:THC, CBDV:THCV, CBDV:THC and CBD:THCV. Alternatively combinations comprising CBD, CBDV, THC and THCV could be used.

A further embodiment of the invention provides a combination of cannabinoids to be used as a pharmaceutical formulation that are packaged for delivery in the form of a gel, a tablet, a liquid, a capsule or for vaporisation. More preferably the combination of cannabinoids to be used as a pharmaceutical formulation are packaged for delivery sublingually or buccally, preferably as a sublingual or buccal spray. Advantageously the pharmaceutical formulation further comprises one or more carrier solvent/s. Preferably the carrier solvents are ethanol and/or propylene glycol. More preferably the ratio of ethanol to propylene glycol is between 4:1 and 1:4. More preferably still the ratio is 1:1.

Favourably the dose is formulated such that a patient is able to titrate their dose. Dose ranges are preferably in the range of between 5 and 25 mg of each cannabinoid, more preferably in the range of 10 to 20 mg of each cannabinoid, preferably in the range of 12 to 14 mg of each cannabinoid more preferably still in the range of 12.5 to 13.5 mg of each cannabinoid.

The administration of a combination of cannabinoids such as THC and CBD could be administered to a patient either at the same time, wherein the cannabinoids would be contained in the same formulation. The cannabinoids could also be administered at separate times for example; a formulation containing CBD could be administered to a patient at a fixed time prior to a formulation containing THC in order to ameliorate some of the side effects of THC, which CBD is known to improve or vice versa. The two cannabinoids could also be administered consecutively to a patient if required.

Preferably the invention provides a combination of cannabinoids, which are present as one or more cannabis based medicine extract/s (CBME/s). In one embodiment the CBME/s are produced by extraction with supercritical or subcritical CO₂. In an additional embodiment the CBME/s are produced by extraction from plant material by volatilisation with a heated gas. Preferably the CBME/s contain all of the naturally occurring cannabinoids in the plant material. Alternatively synthetic or highly purified isolates of the cannabinoids can be used.

According to a second aspect of the present invention there is provided a method of treating a subject with arthritis, which comprises administering the subject a combination of cannabinoids x and y, where x is selected from the group consisting of cannabidiol (CBD) and cannabidivarin (CBDV) and where y is selected from the group consisting of delta-9-tetrahydrocannabinol (THC) and tetrahydrocannabinovarin (THCV), wherein the ratio of x:y by weight is less than or equal to 19:1.

Certain aspects of this invention are further described, by way of example only, with reference to the accompanying drawings in which:

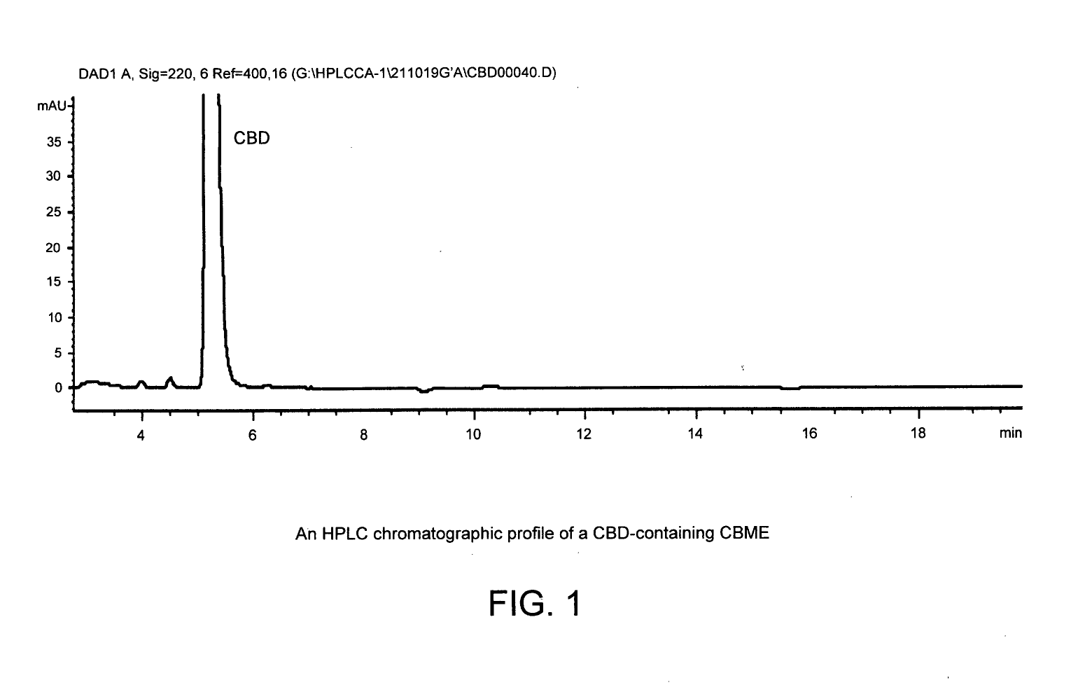

FIG. 1 shows an HPLC chromatographic profile which characterises a CBD-containing cannabis based medicine extract;

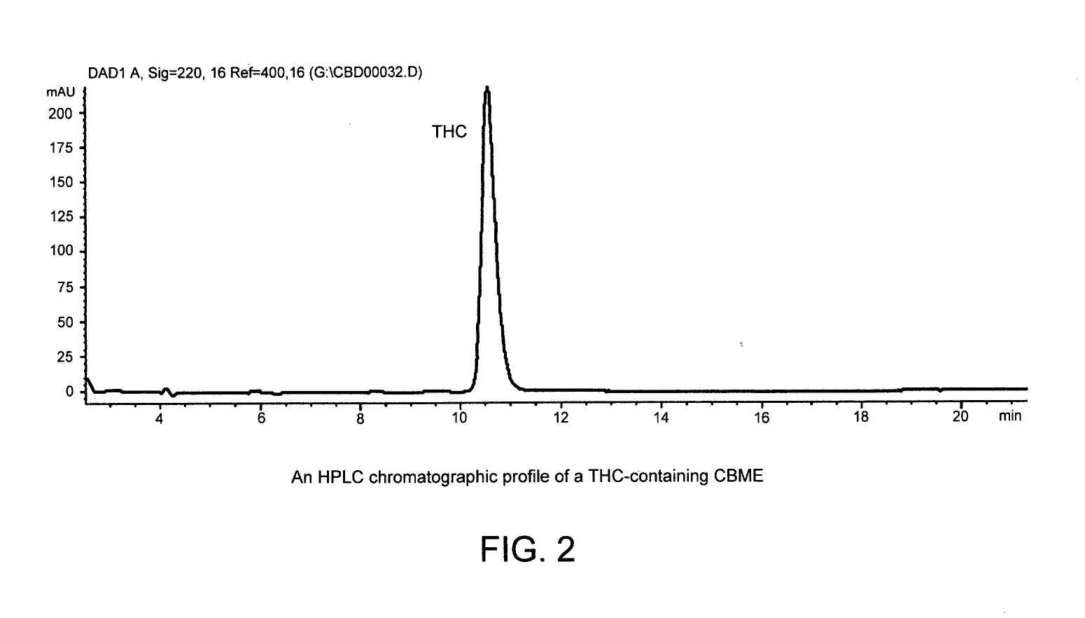

FIG. 2 shows an HPLC chromatographic profile which characterises a THC-containing cannabis based medicine extract; and

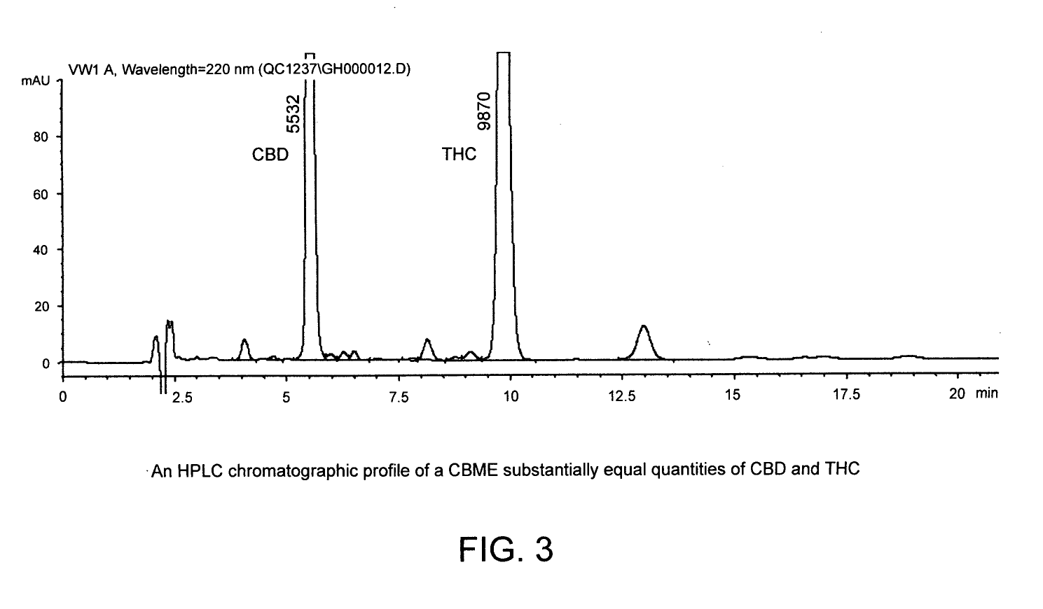

FIG. 3 shows an HPLC chromatographic profile which characterises a cannabis based medicine extract comprising substantially equal quantities of CBD and THC.

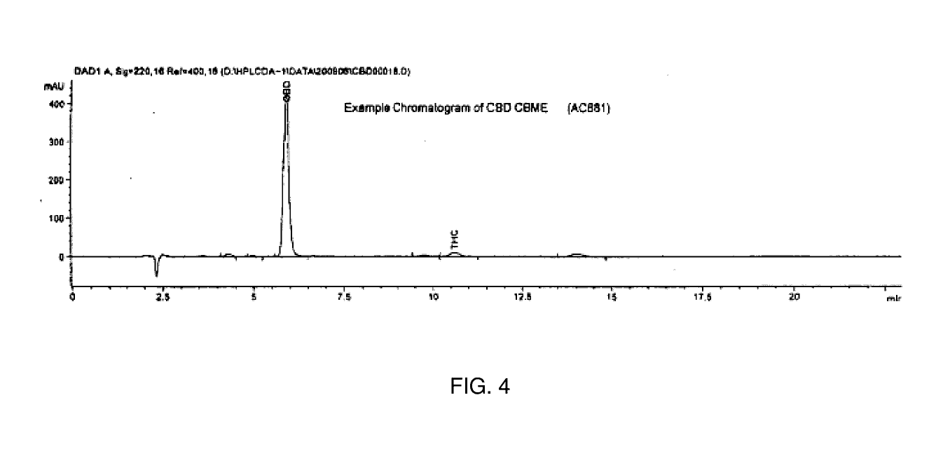

FIG. 4 shows an HPLC chromatographic profile of a CBD-containing cannabis based medicine extract (CBME).

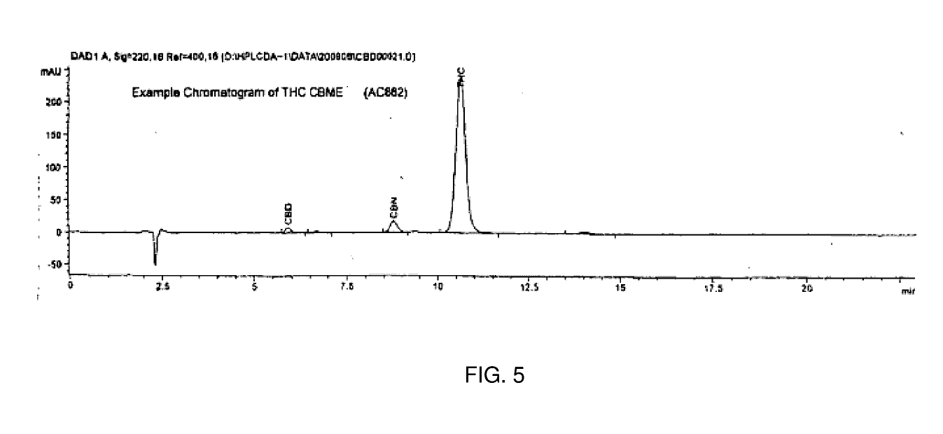

FIG. 5 shows an HPLC chromatographic profile of a THC-containing cannabis based medicine extract (CBME).

### SPECIFIC DESCRIPTION

A cannabis based medicine extract (CBME) was prepared as outlined in Example 1 and contained approximately equal amounts of the cannabinoids THC and CBD and this was administered to patients with chronic rheumatoid arthritis with pain as a secondary condition. The administration of this combination of cannabinoids could possibly reduce the pain and inflammation caused by the rheumatoid arthritis but unexpectedly the cannabis based medicine extract containing approximately equal quantities of THC and CBD also produced a disease modifying effect in the patients with rheumatoid arthritis.

The features of the invention are illustrated further by reference to the following examples, together with the accompanying Figures in which:

FIG. 1 shows an HPLC chromatographic profile of a CBD-containing cannabis based medicine extract (CBME).

FIG. 2 shows an HPLC chromatographic profile of a THC-containing cannabis based medicine extract (CBME).

FIG. 3 shows an HPLC chromatographic profile of a cannabis based medicine extract (CBME) containing substantially equal quantities of CBD and THC.

FIG. 4 shows an HPLC chromatographic profile of a CBD-containing cannabis based medicine extract (CBME).

FIG. 5 shows an HPLC chromatographic profile of a THC-containing cannabis based medicine extract (CBME).

### Example 1

### Preparation of Cannabis Based Medicine Extracts (CBME)

Medicinal cannabis was produced and prepared with reference to the method disclosed in WO 02/064109 (Example 15). The resulting plant material was processed as described in the steps below. The process of manufacture of a High THC or High CBD cannabis based medicine extract is described below:
Medicinal Cannabis (High THC or High CBD) is obtained; followed by
   Chopping to predominantly 2-3 mm; followed by
Heating at 100-150° C. for sufficient time to decarboxylate the acid form of cannabinoids to produce neutral cannabinoids; followed by
   Extraction with a specific volume of liquid carbon dioxide over 6 to 8 hours; followed by
   Removal of CO₂ by depressurisation to recover crude extract; followed by
   Winterisation followed by chilling (−20° c./48 h) to precipitate unwanted waxes; followed by
Removal of unwanted waxy material by cold filtration; followed by
   Removal of ethanol from the filtrate by thin film evaporation under reduced pressure.

The resulting extract is referred to as a cannabis based medicinal drug extract and is also classified as a Botanical Drug Substance according to the US Food and Drug Administration Guidance for Industry Botanical Drug Products.

The quantity of cannabinoid in the CBME can be accurately assessed by way of measurement by HPLC with reference to the method disclosed in WO 02/064109 (Example 16).

An example of an HPLC chromatogram of a CBD-containing CBME produced using a high CBD medicinal cannabis plant extracted with CO₂ is shown in FIG. 1. An example of an HPLC chromatogram of a THC-containing CBME produced using a high THC medicinal cannabis plant extracted with CO₂ is shown in FIG. 2. An example of an HPLC chromatogram containing the relevant ratios of THC and CBD CBMEs is shown in FIG. 3.

The invention has been exemplified with reference to THC and CBD yet it is clear to a man skilled in the art that the pharmacological similarities between THC and THCV and CBD and CBDV are such that similar results could be produced using the cannabinoids THCV and CBDV in place of or in addition to THC and CBD.

### Example 2

### Assessment of the Efficacy of a Cannabis Based Medicine Extract by Way of a Clinical Trial in Human Rheumatoid Arthritis Patients

A seven week, multi-centre, double blind, randomised, parallel group study was undertaken in order to evaluate the efficacy a cannabis based medicine extract on pain in rheumatoid arthritis. The cannabis based medicine extract contained delta-9-tetrahydrocannabinol (THC) at a concentration of 27 mg/ml and cannabidiol (CBD) at a concentration of 25 mg/ml in ethanol:propylene glycol (50:50) excipient. The cannabis based medicine extract was presented in a pump action spray where each activation delivers 100 μl of spray, containing THC (2.7 mg) and CBD (2.5 mg).

The subjects in the study were randomised equally to either the cannabis based medicine extract or a placebo. The placebo matched the appearance, smell and taste of the active formulation, but containing no active components, in ethanol:propylene glycol (50:50) excipient. Again the placebo was presented in a pump action spray where each activation delivers 100 μl of spray.

Patients were screened to determine eligibility at visit 1 and baseline assessments were taken at this time. The patients returned 2 weeks later for visit 2 at which point they were randomised into one of the two groups. The study medication was administered as an evening dose only and patients were asked to titrate their dose until they obtained optimum efficiency.

After 2 weeks titration on the medication the patients returned for visit 3, at this point the patient confirmed the dose that they were to take for the remaining 3 weeks of the study.

The dose of medication that each patient took varied but was in the range of 5-25 mg each of THC and CBD, with the majority of patients receiving between 10 and 20 mg each of THC and CBD. The average dose that each patient titrated to was 13.5 mg THC and 12.5 mg CBD.

After 5 weeks on the study medication the patients returned to make visit 4. All baseline assessments were repeated at this stage.

Efficacy assessments were considered as part of the study. Diary card self-assessments were recorded by each patient on a daily basis for morning pain at rest and on movement, morning stiffness and quality of sleep. Short form McGill Questionnaires were completed at visits 1 and 4 in order to compare changes in intensity of pain, intensity of pain at present, pain at present and global impression of change.

A Disease Activity Score was calculated at visits 1 and 4 from a 28 joint count, erythrocyte sedimentation rate and global disease activity score.

Assessments of the use of rescue analgesia, adverse events, blood chemistry and vital signs were all recorded at visits 1 and 4 in order to consider any changes.

Results:

Some of the data collated from this study is described below.

Comparison of Morning Pain at Rest in Patients with Rheumatoid Arthritis when Administered a Cannabis Based Medicine Extract Containing THC at a Concentration of 27 mg/ml and CBD at a Concentration of 25 mg/ml

The efficacy of a cannabis based medicine extract was assessed as described above and the degree of morning pain at rest was recorded by self assessment on a daily basis. The data was collated and statistical analysis was undertaken. Patients assessed morning pain at rest on a scale of 0 (no pain) to 10 (extremely bad pain). Tables 1 and 2 illustrate the results.

TABLE 1

| | | THC:CBD | |
|---|---|---|---|
| | | (27 mg/ml:25 mg/ml) | Placebo |
| | | (N = 31) | (N = 27) |
| Baseline | Mean | 5.5 | 5.6 |
| | Std Dev | 1.8 | 1.6 |
| | Minimum | 2 | 3 |
| | Median | 5.3 | 5.3 |
| | Maximum | 10 | 9 |
| Week 1 | Mean | 4.6 | 5.2 |
| | Std Dev | 1.6 | 1.6 |
| | Minimum | 1 | 3 |
| | Median | 4.6 | 4.9 |
| | Maximum | 9 | 9 |
| Week 1 - | Mean | −0.9 | −0.4 |
| change from | Std Dev | 1.1 | 1.0 |
| baseline | Minimum | −5 | −3 |
| | Median | −0.6 | −0.4 |
| | Maximum | 1 | 2 |
| Week 2 | Mean | 3.7 | 4.3 |
| | Std Dev | 1.9 | 1.9 |
| | Minimum | 1 | 1 |
| | Median | 3.7 | 4.2 |
| | Maximum | 9 | 10 |
| Week 2 - | Mean | −1.7 | −1.1 |
| change from | Std Dev | 1.8 | 1.6 |
| baseline | Minimum | −6 | −5 |
| | Median | −1.3 | −0.8 |
| | Maximum | 1 | 2 |
| Week 3 | Mean | 3.7 | 4.4 |
| | Std Dev | 1.8 | 1.7 |
| | Minimum | 0 | 0 |
| | Median | 3.6 | 4.3 |
| | Maximum | 8 | 8 |
| Week 3 - | Mean | −1.8 | −1.1 |
| change from | Std Dev | 1.8 | 1.7 |
| baseline | Minimum | −7 | −5 |
| | Median | −1.3 | −0.8 |
| | Maximum | 0 | 2 |
| Week 4 | Mean | 3.5 | 4.4 |
| | Std Dev | 1.8 | 1.9 |
| | Minimum | 0 | 1 |
| | Median | 3.3 | 4.4 |
| | Maximum | 9 | 8 |
| Week 4 - | Mean | −2.0 | −1.0 |
| change from | Std Dev | 1.9 | 1.7 |
| baseline | Minimum | −7 | −5 |
| | Median | −1.6 | −0.8 |
| | Maximum | 1 | 3 |
| Week 5 | Mean | 3.4 | 4.3 |
| | Std Dev | 1.8 | 1.9 |
| | Minimum | 0 | 0 |
| | Median | 3.1 | 4.3 |
| | Maximum | 8 | 8 |
| Week 5 - | Mean | −2.0 | −1.1 |
| change from | Std Dev | 2.0 | 1.9 |
| baseline | Minimum | −7 | −5 |
| | Median | −1.8 | −1.0 |
| | Maximum | 1 | 2 |
| Week 6 | Mean | 3.6 | 4.6 |
| | Std Dev | 1.7 | 0.5 |
| | Minimum | 2 | 4 |
| | Median | 3.0 | 4.9 |
| | Maximum | 6 | 5 |
| Week 6 - | Mean | −2.3 | −0.2 |
| change from | Std Dev | 0.9 | 1.3 |
| baseline | Minimum | −3 | −2 |
| | Median | −2.0 | 0.5 |
| | Maximum | −2 | 1 |
| End Point | Mean | 3.5 | 4.7 |
| | Std Dev | 1.7 | 2.1 |
| | Minimum | 0 | 0 |
| | Median | 3.1 | 4.1 |
| | Maximum | 8 | 9 |
| End Point - | Mean | −2.0 | −0.9 |
| change from | Std Dev | 1.9 | 1.7 |
| baseline | Minimum | −7 | −5 |
| | Median | −1.5 | −0.7 |
| | Maximum | 1 | 2 |

Statistical analysis of this data is shown in Table 2.

TABLE 2

| THC:CBD | | | | | | |
|---|---|---|---|---|---|---|
| (27 mg/ml:25 mg/ml) | | | | | | |
| LS | | Placebo | | | | |
| Mean | s.e. | LS Mean | s.e. | Difference | 95% CI | p-value |
| −2.01 | 0.30 | −0.87 | 0.32 | −1.13 | [−2.02, | 0.013 |
| | | | | | −0.25] | |

The LS Mean figure is the mean change from the baseline adjusted score, a negative difference indicates a benefit.

Tables 1 and 2 demonstrate that the administration of THC:CBD (27 mg/ml:25 mg/ml) to patients suffering pain in rheumatoid arthritis results in a statistically significant reduction in morning pain at rest when compared to the placebo.

Comparison of Quality of Sleep in Patients with Rheumatoid Arthritis when Administered a Cannabis Based Medicine Extract Containing THC at a Concentration of 27 mg/ml and CBD at a Concentration of 25 mg/ml

The efficacy of a cannabis based medicine extract was assessed as described above and the quality of sleep experienced by the patient was recorded by self assessment on a daily basis. The data was collated and statistical analysis was undertaken. Patients assessed quality of sleep on a scale of 0 (very good) to 10 (very bad). Tables 3 and 4 illustrate the results.

TABLE 3

| | | THC:CBD | |
|---|---|---|---|
| | | (27 mg/ml:25 mg/ml) | Placebo |
| | | (N = 31) | (N = 27) |
| Baseline | Mean | 5.7 | 5.8 |
| | Std Dev | 1.9 | 1.8 |
| | Minimum | 2 | 3 |
| | Median | 5.5 | 6.0 |
| | Maximum | 10 | 10 |
| Week 1 | Mean | 4.7 | 5.3 |
| | Std Dev | 1.8 | 1.8 |
| | Minimum | 2 | 2 |
| | Median | 4.9 | 5.4 |
| | Maximum | 8 | 10 |
| Week 1 - | Mean | −1.0 | −0.5 |
| change from | Std Dev | 1.7 | 1.1 |
| baseline | Minimum | −6 | −3 |
| | Median | −0.9 | −0.3 |
| | Maximum | 2 | 2 |
| Week 2 | Mean | 3.6 | 4.6 |
| | Std Dev | 2.1 | 1.7 |
| | Minimum | 0 | 2 |
| | Median | 3.5 | 4.4 |
| | Maximum | 10 | 9 |
| Week 2 - | Mean | −2.1 | −1.1 |
| change from | Std Dev | 2.0 | 1.9 |
| baseline | Minimum | −8 | −7 |
| | Median | −1.7 | −0.8 |
| | Maximum | 1 | 2 |
| Week 3 | Mean | 3.8 | 4.4 |
| | Std Dev | 2.2 | 1.9 |
| | Minimum | 0 | 0 |
| | Median | 3.6 | 4.4 |
| | Maximum | 9 | 8 |
| Week 3 - | Mean | −2.0 | −1.4 |
| change from | Std Dev | 2.0 | 1.8 |
| baseline | Minimum | −7 | −6 |
| | Median | −1.6 | −1.1 |
| | Maximum | 1 | 1 |
| Week 4 | Mean | 3.5 | 4.5 |
| | Std Dev | 2.2 | 2.1 |
| | Minimum | 0 | 1 |
| | Median | 3.4 | 4.0 |
| | Maximum | 9 | 9 |
| Week 4 - | Mean | −2.3 | −1.4 |
| change from | Std Dev | 2.2 | 2.1 |
| baseline | Minimum | −9 | −6 |
| | Median | −1.9 | −0.9 |
| | Maximum | 2 | 2 |
| Week 5 | Mean | 3.3 | 4.5 |
| | Std Dev | 2.2 | 2.2 |
| | Minimum | 0 | 0 |
| | Median | 3.0 | 4.3 |
| | Maximum | 8 | 9 |
| Week 5 - | Mean | −2.5 | −1.3 |
| change from | Std Dev | 2.2 | 2.1 |
| baseline | Minimum | −9 | −6 |
| | Median | −2.1 | −1.2 |
| | Maximum | 1 | 2 |
| Week 6 | Mean | 2.6 | 5.1 |
| | Std Dev | 1.8 | 1.6 |
| | Minimum | 1 | 4 |
| | Median | 2.2 | 4.9 |
| | Maximum | 5 | 7 |
| Week 6 - | Mean | −2.2 | −0.2 |
| change from | Std Dev | 1.1 | 1.7 |
| baseline | Minimum | −3 | −3 |
| | Median | −2.3 | 0.3 |
| | Maximum | −1 | 1 |
| End Point | Mean | 3.4 | 4.6 |
| | Std Dev | 2.2 | 2.2 |
| | Minimum | 0 | 1 |
| | Median | 3.5 | 4.0 |
| | Maximum | 8 | 10 |
| End Point - | Mean | −2.3 | −1.1 |
| change from | Std Dev | 2.2 | 2.0 |
| baseline | Minimum | −9 | −5 |
| | Median | −1.8 | −0.9 |
| | Maximum | 1 | 2 |

Statistical analysis of this data is shown in Table 4.

TABLE 4

| THC:CBD | | | | | | |
|---|---|---|---|---|---|---|
| (27 mg/ml:25 mg/ml) | | | | | | |
| LS | | Placebo | | | | |
| Mean | s.e. | LS Mean | s.e. | Difference | 95% CI | p-value |
| −2.31 | 0.35 | −1.14 | 0.38 | −1.17 | [−2.00, | 0.027 |
| | | | | | −0.14] | |

The LS Mean figure is the mean change from the baseline adjusted score, a negative difference indicates a benefit.

Tables 3 and 4 demonstrate that the administration of THC:CBD (27 mg/ml:25 mg/ml) to patients suffering pain in rheumatoid arthritis results in an improved quality of sleep when compared to the placebo.

Comparison of Disease Activity Score in Patients with Rheumatoid Arthritis when Administered a Cannabis Based Medicine Extract Containing THC at a Concentration of 27 mg/ml and CBD at a Concentration of 25 mg/ml

The efficacy of a cannabis based medicine extract was assessed as described above and the Disease Activity Score for each patient was determined at visits 1 and 4. The data was collated and statistical analysis was undertaken. Tables 5 and 6 illustrate the results.

TABLE 5

| | | THC:CBD | |
|---|---|---|---|
| | | (27 mg/ml:25 mg/ml) | Placebo |
| | | (N = 31) | (N = 27) |
| Visit 1 | Mean | 5.88 | 6.00 |
| | Std Dev | 0.95 | 1.03 |
| | Minimum | 4.6 | 3.8 |
| | Median | 5.70 | 6.00 |
| | Maximum | 7.8 | 7.8 |
| Visit 4 | Mean | 5.00 | 5.90 |
| | Std Dev | 1.09 | 1.10 |
| | Minimum | 3.0 | 4.0 |
| | Median | 4.90 | 5.80 |
| | Maximum | 7.1 | 8.2 |
| Change from | Mean | −0.85 | −0.16 |
| Visit 1 | Std Dev | 0.81 | 0.98 |
| | Minimum | −2.7 | −3.0 |
| | Median | −0.70 | 0.05 |
| | Maximum | 0.5 | 1.3 |

Statistical analysis of this data is shown in Table 6.

TABLE 6

| THC:CBD | | | | | | |
|---|---|---|---|---|---|---|
| (27 mg/ml:25 mg/ml) | | | | | | |
| LS | | Placebo | | | | |
| Mean | s.e. | LS Mean | s.e. | Difference | 95% CI | p-value |
| −0.88 | 0.16 | −0.03 | 0.17 | −0.76 | [−1.23, | 0.002 |
| | | | | | −0.28] | |

The LS Mean figure is the mean change from the baseline adjusted score, a negative difference indicates a benefit.

Tables 5 and 6 demonstrate that the administration of THC:CBD (27 mg/ml:25 mg/ml) to patients suffering pain in rheumatoid arthritis results in an improved Disease Activity Score when compared to the placebo.

The use of a mixture of THC and CBD, where the cannabinoids are in approximately equal quantities when provided to patients with pain associated with rheumatoid arthritis resulted in a decrease in morning pain at rest. The quality of sleep that was experienced by the patients provided with the mixture of equal quantities of THC and CBD was also shown to improve. The patients who were provided with the medication also experienced a decrease in their pain at present as recorded from a questionnaire. Most significantly of all was the effect the medication had on the patients Disease Activity Score.

The Disease Activity Score is a method used to measure the degree of rheumatoid arthritis experienced by a patient. It involves determination of how swollen and tender 28 different joints are. A blood test is also used as part of the Disease Activity Score to measure the erythrocyte sedimentation rate. This rate is a lab method for determining an acute phase response to inflammation. A global disease activity score based on how the patient is feeling also contributes to an overall figure that is calculated. A composite score of greater than 3.7 is considered to be high.

The significance of the findings of the present invention that the use of an approximately 1:1 combination of THC and CBD are able to decrease the Disease Activity Score in patients with rheumatoid arthritis is great.

### REFERENCES

Formukong E. A., Evans A. T. and Evans F. J. (1988) Analgesic and Antiinflammatory activity of constituents of Cannabis sativa L. Inflammation 12(4), 361-371
Holdcroft A. et al. (1997a) Pain relief with oral cannabinoids in familial Mediterranean fever. Anaesthesia 52(5), 483-6
Holdcroft A. et al. (1997b) Clinical trial experience with cannabinoids. Pharm. Sci. 3, 546-550
Klein T. W., Newton C. and Friedman H. (1998) Immunol. Today 19, 373-380

1.-42. (canceled)

43. A method for treatment of arthritis comprising administering to a subject in need thereof a pharmaceutical formulation comprising a therapeutically effective amount of a combination of cannabidiol (CBD) and tetrahydrocannabinol (THC), wherein the ratio of CBD:THC by weight is substantially 1:1.

44. The method of claim 43, wherein the arthritis is osteoarthritis.

45. The method of claim 43, wherein the arthritis is rheumatoid arthritis.

46. The method of claim 43, wherein the treatment of arthritis comprises treatment of pain in arthritis.

47. The method of claim 43, wherein the treatment of arthritis is comprises treatment of inflammation in arthritis.

48. The method of claim 43, wherein the treatment of arthritis comprises improvement in quality of sleep in arthritis.

49. The method of claim 43, wherein the treatment of arthritis comprises disease modification of arthritis.

50. The method of claim 43, wherein the treatment of arthritis comprises treatment of one or more symptoms of arthritis and disease modification of arthritis.

51. The method of claim 50, wherein the one or more symptoms are pain, inflammation or lack of sleep.

52. The method of claim 43, wherein the ratio of CBD:THC is 0.93:1.

53. The method of claim 43, wherein the pharmaceutical formulation is packaged for delivery sublingually or buccally.

54. The method of claim 43, wherein the pharmaceutical formulation is in the form of a gel or gel spray, a tablet, a liquid, a capsule or for vaporization.

55. The method of claim 43, wherein the pharmaceutical formulation further comprises one or more carrier solvent(s).

56. The method of claim 55, wherein the carrier solvent(s) are ethanol and/or propylene glycol.

57. The method of claim 56, wherein the ratio of ethanol and propylene glycol is between 4:1 and 1:4.

58. The method of claim 56, wherein the ratio of ethanol and propylene glycol is 1:1.

59. The method of claim 43, wherein the pharmaceutical formulation is in a titratable dosage form.

60. The method of claim 43, wherein the therapeutically effective amount taken by the subject is a dose in the range of 5-25 mg per day of each of CBD and THC.

61. The method of claim 43, wherein CBD is administered separately, simultaneously or sequentially to THC.

62. The method of claim 43, wherein the CBD and THC are present as a cannabis based medicine extract (CBME), or wherein the CBD and THC are derived from one or more CBME(s).

63. The method of claim 62, wherein the formulation comprises a combination of:
a) a cannabis based medicinal extract which comprises THC at more than 90% of the total cannabinoid content in the extract; and
b) a cannabis based medicinal extract which comprises CBD at more than 90% of the total cannabinoid content in the extract.

64. The method of claim 62, wherein the CBME(s) are produced by extraction with supercritical or subcritical CO₂.

65. The method of claim 62, wherein the CBME(s) are produced by contacting plant material with a heated gas at a temperature which is greater than 100° C., sufficient to volatilize one or more of the cannabinoids in the plant material to form a vapor, and condensing the vapor to form an extract.

66. The method of claim 62, wherein the CBME(s) comprise all the naturally occurring cannabinoids in the said plant(s).

67. The method of claim 43, wherein the CBD and THC are substantially pure.

68. The method of claim 43, wherein the CBD and THC are synthetic.

69. The method of claim 62, wherein the CBD-containing CBME is characterized by a chromatographic profile as illustrated in FIG. 4, where the retention time of the CBD is between 5.4 and 6.4 minutes.

70. The method of claim 62, wherein the THC-containing CBME is characterized by a chromatographic profile as illustrated in FIG. 5, where the retention time of the THC is between 9.6 and 10.6 minutes.

71. The method of claim 62, wherein the CBME containing THC and CBD is characterized by a chromatographic profile as illustrated in FIG. 3.
